(19) United States
(12) Patent Application Publication
(10) Pub. No.: US 2026/0076916 A1
(43) Pub. Date: Mar. 19, 2026

(54) METHYLPHENIDATE MODIFIED RELEASE FORMULATIONS

(71) Applicant: Granules Pharmaceuticals Inc., Chantilly, VA (US)

(72) Inventors: Sudhir R. Gorukanti, Ashburn, VA (US); Rajan Sharma Bhattarai, Herndon, VA (US); Mallikharjun Vasireddy, Manassas, VA (US); Chintan Desai, Manassas, VA (US)

(21) Appl. No.: 19/333,698

(22) Filed: Sep. 19, 2025

Related U.S. Application Data
(60) Provisional application No. 63/696,458, filed on Sep. 19, 2024.

(51) Int. Cl. A61K 9/50; A61K 9/28; A61K 31/137; A61K 31/4458; A61K 47/10; A61K 47/38

(52) U.S. Cl. CPC A61K 9/5042; A61K 9/282; A61K 9/2846; A61K 9/501; A61K 9/5015; A61K 9/5026; A61K 9/5073; A61K 31/137; A61K 31/4458; A61K 47/10; A61K 47/38

(57) ABSTRACT

The present invention relates to therapeutic compositions and methods for the treatment of attention deficit disorder (ADD), or attention deficit hyperactivity disorder (ADHD). The present invention more particularly relates to a modified-release pharmaceutical formulation designed to improve the delivery and efficacy of central nervous system (CNS) stimulants. The composition consists of a drug-containing core with a CNS stimulant and pharmaceutically acceptable excipients. This core is coated with a sustained-release (SR) layer comprising one or more polymers selected from water-insoluble and water-insoluble pH-dependent polymers, and one or more pharmaceutically acceptable excipients which regulates the gradual release of the drug. Surrounding the SR layer is a delayed-release (DR) outer layer that controls the onset of drug release. This approach aims to enhance symptom control, reduce dosing frequency, and improve patient compliance in the treatment of ADD and ADHD.

### FIELD OF INVENTION

The present invention relates to therapeutic compositions and methods for the treatment of attention deficit disorder (ADD), or attention deficit hyperactivity disorder (ADHD).

### BACKGROUND OF THE INVENTION

Methylphenidate hydrochloride, a central nervous system (CNS) stimulant. It is chemically known as d,l (racemic) methyl α-phenyl-2-piperidineacetate hydrochloride. Its structural formula is

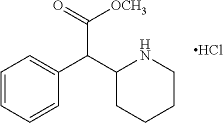 [SMILES: COC(=O)C(c1ccccc1)C1CCCCN1 ; Cl]

Attention deficit hyperactivity disorder (ADHD) is presently a difficult disorder to diagnose. The core symptoms of ADHD in children include inattention, hyperactivity, and impulsivity. ADHD children may experience significant functional problems, such as school difficulties, academic underachievement, poor relationships with family and peers, and low self-esteem. Adults with ADHD often have a history of losing jobs, impulsive actions, substance abuse, and broken marriages. ADHD often goes undiagnosed if not caught at an early age and affects many adults who may not be aware of the condition. ADHD has many look-alike causes (family situations, motivations) and co-morbid conditions (depression, anxiety, and learning disabilities) are common.

Central nervous system stimulants widely used in the treatment of Attention Deficit Disorder (“ADD”), Methylphenidate HCl is also used to treat a related disorder, Attention Deficit Hyperactivity Disorder (“ADHD”), in which symptoms of hyperactivity are present along with the symptoms of ADD. The drug is additionally used in the symptomatic treatment of narcolepsy, depression etc. one of the difficulties in treating ADHD and other CNS stimulant responsive conditions is instilling and maintaining effective concentration in patients throughout the day, particularly in the morning hours when cognitive abilities and concentration are required for school or work, and in the late afternoon or evening when students frequently do homework. Early formulations relied on twice-daily delivery of an immediate-release formulation, which caused compliance issues.

Methylphenidate Hydrochloride, a scheduled II controlled substance, is currently marketed as a mild central nervous system (CNS) stimulant and the treatment of attention deficit disorder (ADD) and attention deficit hyperactivity disorder (ADHD) in adults and children has been described [see, Focalin®, Concerta®, Ritalin®, Daytrana® and Metadate® product literature]. The drug is well absorbed throughout the gastrointestinal tract. However, it has an extremely short half-life, which necessitates a multi-dose treatment regimen for conventional (immediate release) dosage forms such as currently available 5, 10, and 20 mg tablets. Due to high Cmax, oral administration of 10 and 20 mg Ritalin® is reported to result in notable side effects such as anorexia, weight loss, dizziness, etc. Furthermore, it requires the hyperactive children to be dosed in school thus causing hardship to school authorities as well as parents. The drawback of methylphenidate is that it also produces a euphoric effect when administered intravenously or through inhalation, thus presenting a high potential for substance abuse.

U.S. Pat. No. 5,908,850 assigned to Celgene Corporation discloses a method for treating children with the above disability to be treated using a sustained release dosage form containing d-threo-methylphenidate or pharmaceutically acceptable salts thereof thus minimizing hyperactivity and side effects. However, it does not address how it avoids dosing in school, thereby minimizing potential drug abuse.

Release controlled formulation refers to a drug delivery system used to release a biologically active ingredient such as a drug over in a controlled manner over a long period of time, e.g. 6 to 24 hours, such as in a sustained manner. The release controlled formulation is advantageous that it can be taken less frequently and it keeps steadier levels of the drug in the bloodstream. Further, sustained-release formulations that can be taken once a day are preferred since the patient frequently forgets to take the medicine at the lunch hour or does not want to be known to have the disorder.

Sustained release (SR) dosage forms are important in the search for better therapy because they increase patient compliance while decreasing the frequency of adverse drug reactions. sustained release formulations aim to prolong the duration of pharmacologic effect beyond what is typically observed following the administration of immediate release dose forms. Compared to similar short acting, instant release preparations, such longer periods of responsiveness offer numerous therapeutic benefits. Longer lasting medication formulations also often increase patient compliance by preventing missed doses due to patient forgetfulness.

Some existing sustained-release techniques employ a multiparticulate modified release system including a combination of immediate-release particles and modified-release particles of methylphenidate. The immediate-release particles are formed without a polymer coating on the drug layer, so that the drug is released immediately.

Therefore, a composition for release control is required that can stabilize the pattern of methylphenidate release, with the drug's release being delayed for four to twelve hours before being delivered in a controlled sustained way.

The current invention discloses numerous compositions: a single dose can be taken in the evening, or at any time of day one retires for an extended period of sleep, and the drug's release is delayed for 4 to 12 hours before being released in a regulated or sustained manner. According to the disclosure, the lag time can be changed by the formulation's design to be 4, 5, 6, 7, 8, 9, 10, 11, or 12 hours, or intermediate intervals within the range.

U.S. Pat. No. 9,498,447 B2 discloses a treatment of attention deficit hyperactivity disorder, using a solid, oral pharmaceutical composition comprising: a single population of beads comprising: Methylphenidate core; a sustained release layer enclosing the Methylphenidate core, wherein the sustained release layer comprises a water-insoluble and water-permeable polymer, a water soluble polymer, a hydrophobic plasticizer, and a hydrophobic binder; and a delayed release layer enclosing the sustained release layer.

The current inventors have created alternative modified release pharmaceutical formulations of methylphenidate and a process for preparing them that are more commercially viable, less difficult, cost-effective and are easier to commercialize. One such approach produces a modified release composition that achieves the desired dissolution profile. Another such approach is free of water-soluble and/or water-permeable polymers, as well as binders in the sustained release layer. These compositions demonstrated the expected dissolution profile and bioavailability, which were also shown to be equivalent to commercially available methylphenidate formulations, such as Jornay PM®.

Further, in the current marketed formulations, the use of water-soluble pore former in the controlled release layer would lead to unwanted premature release of the formulation in stomach. Therefore, a novel formulation using water insoluble pH dependent pore former in the Sustained-release layer was developed to assure no premature release in stomach and minimize the associated adverse effects like early awakening and insomnia.

### SUMMARY OF THE INVENTION

In one aspect, the present invention provides modified release formulations of CNS stimulant, that are administered once daily at evening or bedtime.

In another aspect the present invention provides a modified release composition comprising: i) a core comprising CNS stimulant and one or more pharmaceutically acceptable excipients; ii) an inner sustained release (SR) layer coated over the drug containing core, the sustained release layer comprising one or more polymers selected from water insoluble and water insoluble pH dependent polymers and one or more pharmaceutically acceptable excipients; and iii) an outer delayed release (DR) layer enclosing the sustained release layer.

In another aspect, the current disclosure's compositions provide modified release formulations for treating diseases or conditions that respond to CNS stimulants. Such illnesses include, but are not limited to, ADD, ADHD, narcolepsy, excessive daytime drowsiness, major depressive disorder, bipolar depression, negative symptoms of schizophrenia, chronic fatigue, chemotherapy-induced weariness, and binge eating disorder. The formulations are successful in treatment the adult, pediatric, and adolescent populations who require such care.

In another aspect, the present invention provides a Modified release (MR) composition comprising; a core, a Sustained release layer (SR), and optionally a Delayed release (DR) layer, wherein the compositions disclosed herein can be in the form of coated beads/Pellets.

In another aspects, the present invention provides a coated bead comprising: (i) a granule comprising an active pharmaceutical ingredient, a central nervous system stimulant (e.g., the granule can comprise a granule substrate in admixture with the active pharmaceutical ingredient or the active pharmaceutical ingredient could be coated over the granule substrate); (ii) an inner layer coated over the granule, the inner layer being configured to permit substantially sustained release of the active pharmaceutical ingredient comprised therein and (iii) an outer layer coated over an inner layer, the outer layer being present in an amount sufficient to substantially delay release of the active pharmaceutical ingredient until after the drug is administered 4 to 12 hours.

Another aspect of the present invention is a modified release composition that includes CNS stimulants and one or more excipients admixed with one or more polymers, to form sustained release matrix granules which is further processed and enclosed by an outer delayed release layer

In another aspect, the present invention provides a modified release composition comprising i) drug-coated beads or pellets, which can contain CNS stimulants and one or more excipients coated on inert pellets or beads; ii) an inner sustained release layer coated over drug-coated beads or pellets; and iii) an outer delayed release layer enclosing the sustained release layer.

In another aspect, the present invention provides a modified release composition comprising; a core, a sustained release coating, and a delayed release (DR) coating, wherein the compositions disclosed herein can be in the form of coated beads/Pellets, wherein the coated bead/Pellets can be compressed in to tablet or minitablet form.

In another aspect, the present invention provides a modified release composition comprising; a core, a sustained release coating, and a delayed release (DR) coating, wherein the compositions disclosed herein can be in the form of coated beads/Pellets, wherein the coated beads/Pellets can then be apportioned in single dose amounts into water-soluble gelatin capsules, or into a liquid or gel suspension for administration.

In another aspect, the present invention provides modified release formulations of CNS stimulants in the form of capsules containing coated beads, pellets, or minitablets, where the coated beads, pellets, or minitablets are coated with an inner, sustained release layer coated over a drug-containing core and an outer, delayed release coating, which delays drug release for 4 to 12 hours.

In another aspect, the present invention provides modified release formulations of CNS stimulant, that are capsules containing coated beads/Pellets (or) minitablets, wherein the coated beads/Pellets or mini tablets are coated with an outer, delayed release coating and an inner, sustained release layer coating over a drug containing core. As the outer delayed layer of the composition dissolves, the sustained release layer begins to lose some of its integrity and the drug starts to release slowly.

In another aspect, the present invention provides a modified release composition comprising: a core containing CNS stimulant and one or more pharmaceutically acceptable excipients or the core can consist essentially of the stimulant and one or more excipients coated on an inert pellet/bead, a sustained release layer coated over drug containing core and a delayed release layer, wherein the disclosed composition is prepared into a suitable dosage form with one or more pharmaceutically excipients.

In yet another aspect, the modified release compositions of the present invention can also be described as solid, oral pharmaceutical compositions including a core comprising a therapeutic amount of a CNS stimulant and at least one pharmaceutically acceptable excipient, wherein the core is substantially free of a disintegrant or pore-forming agent; a sustained release layer coated over the drug core; and a delayed release layer enclosing the sustained release layer.

In another aspect, the present invention provides a modified release composition comprising: a core containing CNS stimulant and one or more pharmaceutically acceptable excipients, a sustained release layer comprising water insoluble polymer and one or more pharmaceutical acceptable excipients and a delayed release layer comprising one or more pH dependent polymers and one or more pharmaceutically acceptable excipients.

In yet another aspect, the present invention provides a modified release composition comprising: a core containing CNS stimulant and one or more pharmaceutically acceptable excipients, a sustained release layer coated over the drug containing core, wherein sustained release layer is free from water soluble polymer, and substantially free from a binder and a delayed release layer enclosing the sustained release layer.

In another aspect, the present invention provides a modified release composition comprising: a core containing CNS stimulant and one or more pharmaceutically acceptable excipients, a sustained release layer comprising a water insoluble polymer, optionally a plasticizer, and one or more pharmaceutically acceptable excipients; and a delayed release layer comprising one or more enteric polymers and one or more pharmaceutically acceptable excipients, wherein the said composition can be compressed into a suitable dosage form like tablets or minitablets.

In yet another aspect, the present invention provides a modified release composition comprising: a core containing CNS stimulant and one or more pharmaceutically acceptable excipients or the core can consist essentially of the stimulant and one or more excipients coated on inert beads or pellets, a sustained release layer coated over drug containing core, wherein sustained release layer comprising a water insoluble polymer, optionally a plasticizer, and one or more pharmaceutically acceptable excipients; and a delayed release layer enclosing the sustained release layer, wherein delayed release layer comprising one or more enteric polymers, optionally a plasticizer and one or more pharmaceutically acceptable excipients, wherein said single multilayer coated beads are formulated into an oral solid pharmaceutical compositions, preferably in the form of a capsule.

In yet another aspect, the current innovators have developed a one-of-a-kind coated bead as part of a new, changed releasing composition. The current coated bead of compositions is thought to be extremely beneficial because it has a controlled or sustained long duration of action (e.g., 16 hours or more) after reaching steady state in the subject, as well as a delayed release of 4 to 12 hours after the drug is administered.

In yet another aspect, the present invention is thought to be very beneficial that the current coated bead of modified release pharmaceutical compositions uses water-insoluble polymers and water-insoluble pH-dependent pore formers in the sustained release layer rather than water-soluble and water-permeable polymers, which simplifies manufacturing costs and other related issues, further the usage of water insoluble pH dependent pore former in the sustained-release layer blocks the premature release of drug in the stomach and assures the release of drug at target site after 8 hours of dosing.

### DETAILED DESCRIPTION OF THE INVENTION

In one embodiment, the present invention provides modified release solid oral pharmaceutical compositions comprising CNS stimulant, that are administered once daily at evening or bedtime.

In another embodiment, the present invention provides modified release compositions comprising coated beads/pellets comprising a central nervous system stimulant which can be generally defined as a chemical entity that affects the dopamine or norepinephrine neural pathways.

The active ingredients include CNS stimulants, which are beneficial in the treatment of ADD and ADHD, as well as other diseases involving dopamine or norepinephrine neural pathways. The active ingredients include, but are not limited to, active isomers of amphetamines and amphetamine salts, as well as mixed amphetamine salts, including dextroamphetamine salts, and methylphenidate, its active salts and known conjugates, all of which can be used as racemic mixtures or pure isomers such as d-threomethylphenidate. The disclosed formulations may additionally contain one or more prodrugs of CNS stimulants, including but not limited to amino acid conjugated active substances such as l-lysine-d-amphetamine.

In yet another embodiment, the preferred pharmaceutically active ingredients include, but are not limited to, methylphenidate, its active salts and known conjugates, or combinations of them; amphetamine, dextroamphetamine, and the active isomers of amphetamines and amphetamine salts, including dextroamphetamine salts. These ingredients can be used as prodrugs, pharmaceutical salts, or mixed pharmaceutical salts of any of the ingredients alone or in combination.

In yet another embodiment, disorders or conditions that can be treated using the present Modified release solid oral pharmaceutical compositions containing coated beads/pellets include, but are not limited to attention deficit disorder (ADD), attention deficit hyperactivity disorder (ADHD), excessive daytime sleepiness, major depressive disorder, bipolar depression, negative symptoms in schizophrenia, chronic fatigue, fatigue associated with chemotherapy or binge eating disorder. Attention deficit disorders are characterized by hyperactive, impulsive, or inattentive symptoms that impede social, academic, or occupational functioning. They are frequently present in two or more environments, such as school (or job) and home.

In yet another embodiment, the current disclosure describes therapeutic compositions for the treatment of attention deficit disorder (ADD), attention deficit hyperactivity disorder (ADHD), and other conditions or disorders that respond to CNS stimulants by providing dosage forms that deliver a therapeutic amount of active drug in a delayed and controlled release pattern, allowing the drug to be maintained throughout the active portion of the day. A therapeutic amount is recommended for pediatric patients, including teenagers, as well as adults, upon waking up and during the morning, as well as throughout the afternoon hours when work or homework must be completed.

In yet another embodiment, the current invention provides modified release solid oral pharmaceutical formulations including a stimulant. Stimulant medications (e.g., methylphenidate, amphetamines, and conjugates & prodrugs thereof) are frequently given to treat patients with ADHD. According to the National Institute of Health, all stimulants increase dopamine levels in the brain. Dopamine is a brain molecule (or neurotransmitter) linked to pleasure, movement, and attention. Stimulants exert their therapeutic impact by slowly and steadily increasing dopamine levels, which are identical to what the brain produces naturally.

In another embodiment, amphetamines effective in the disclosed formulations comprise amphetamine and its isomers such as dextroamphetamine, d,l-amphetamines, and their pharmaceutically acceptable salts, such as sulfate, saccharate, and aspartate salts, for example. Amphetamines are sympathomimetic amines that stimulate the central nervous system (CNS). Dextroamphetamine is the dextro isomer of the sympathomimetic amine d,l-amphetamine sulfate. Dextroamphetamine is the chemical name for d-alpha-methylphencthylamine. Dextroamphetamine or alternative pharmaceutically approved salts of dextroamphetamine can be used in the practice of this disclosure.

In another embodiment, Methylphenidate is useful in the disclosed formulations. Preferred pharmaceutically active substances include, but are not limited to, methylphenidate and its active salts. Methylphenidate is another CNS, (CNS) stimulant approved by the FDA in 1955 for hypeactivity. Methylphenidate [dl-threo-methyl-2-phenyl-2-(2-piperidyl)acetate] is the most often used psychostimulant to treat hyperactivity and attention deficit disorder. Methylphenidate can be administered in a racemic combination of dextro and levo conformations, or as the pure dextro isomer. Methylphenidate contains two chiral centers and can thus be processed to enrich the d threo isomer. The present disclosure further contemplates the use of pharmaceutically approved methylphenidate salts, such as methylphenidate hydrochloride.

Throughout this specification, the term “coated over” (or the functional equivalent thereof) is used to describe a first layer of material disposed exteriorly with respect to a second layer of material. It should be clearly understood that, in such a case, the first layer layer of material may be directly coated over (i.e., in contacting relation with) the second layer of material or indirectly coated over (i.e., in non-contacting relation with) the second layer of material. An example of the “indirectly coated over” would be when the first layer of material and the second layer of material has disposed between them one or more intermediate layers of material. The point is the term “coated over” (or the functional equivalent thereof), when used on its own encompasses both “directly coated over” and “indirectly coated over” described above.

In certain embodiments, at least the delayed release layer includes one or more polymers such as an acrylic polymer, acrylic copolymer, methacrylic polymer or methacrylic copolymer, including but not limited to Eudragit® L100, Eudragit® L100-55, Eudragit® L 30 D-55, Eudragit® 5100, Eudragit® 4135F, Eudragit® RS, acrylic acid and methacrylic acid copolymers, methyl methacrylate, methyl methacrylate copolymers, ethoxyethyl methacrylates, cyanoethyl methacrylate, aminoalkyl methacrylate copolymer, polyacrylic acid, polymethacrylic acid, methacrylic acid alkylamine copolymer, polymethyl methacrylate, polymethacrylic acid anhydride, polymethacrylate, polyacrylamide, polymethacrylic acid anhydride and glycidyl methacrylate copolymers, an alkylcellulose such as ethylcellulose, methylcellulose, calcium carboxymethyl cellulose, polyvinyl acetate phthalate, polyester, waxes, shellac, zein, or the like.

In certain embodiments, a plasticizer is also included in the oral dosage form. Plasticizers suitable for use in the present invention include, but are not limited to, low molecular weight polymers, oligomers, copolymers, oils, small organic molecules, low molecular weight polyols having aliphatic hydroxyls, ester-type plasticizers, glycol ethers, poly(propylene glycol), multi-block polymers, single block polymers, low molecular weight poly(ethylene glycol), citrate ester-type plasticizers, triacetin, propylene glycol and glycerin. Such plasticizers can also include ethylene glycol, 1,2-butylene glycol, 2,3-butylene glycol, styrene glycol, diethylene glycol, triethylene glycol, tetraethylene glycol and other poly(ethylene glycol) compounds, monopropylene glycol monoisopropyl ether, propylene glycol monoethyl ether, ethylene glycol monoethyl ether, diethylene glycol monoethyl ether, sorbitol lactate, ethyl lactate, butyl lactate, ethyl glycolate, dibutyl sebacate, acetyltributylcitrate, triethyl citrate, acetyl triethyl citrate, tributyl citrate and allyl glycolate.

In certain embodiments, one or more suitable water insoluble polymers and water insoluble, PH dependent polymers are selected to be used in the invention, which include, but are not limited to, cellulosic polymers such as Ethyl cellulose, hydroxypropyl methylcellulose phthalate, and hydroxypropyl methylcellulose acetate succinate, a cellulose ester, an acrylate-based polymer or copolymer, i.e., an acrylic acid or acrylic acid ester polymer or copolymer (an “acrylate” polymer) and polyvinyl acetate-based polymers.

Exemplary cellulose esters include, for example, cellulose acetate, cellulose acetate propionate (CAP), cellulose acetate butyrate (CAB), cellulose acetate phthalate, cellulose propionate (CP), cellulose butyrate (CB), cellulose propionate butyrate (CPB), cellulose diacetate (CDA), cellulose triacetate (CTA), cellulose acetate trimellitate (CAT) or the like

In one embodiment, the modified release composition of the present invention can be formulated as an oral formulation in the form of capsules, tablets, beads, granules, powders, sachets, pouches, sprinkles, multiunit particulate systems (MUPS), pellets, spheres, particles or mini-tablets, or a liquid formulation such as a syrup, an emulsion, a suspension, and the like.

In another embodiment of the Modified release compositions of the present disclosure is a dosage form that includes a capsule enclosing a single population of beads or minitablets that include a core and two or more coatings surrounding the core. The inner core is a bead or minitablet containing an API and one or more excipients.

The term beads, particles, pellets, micro particles as used herein include a plurality of core pellets that in certain embodiments are substantially spherical beads.

In another embodiment, the present invention provides a modified release composition comprising; a core, a sustained release layer, and optionally a delayed release (DR) layer, wherein the compositions disclosed herein can be in the form of coated beads/Pellets.

In another embodiment, the present invention provides a modified release (MR) composition comprising i) a core component comprising CNS stimulant and one or more pharmaceutically acceptable excipients ii) sustained release (SR) layer and iii) a delayed release (DR) layer.

In yet another embodiment, the present invention provides a modified release composition comprising i) a core component comprising CNS stimulant and one or more pharmaceutically acceptable excipients or the core can consist essentially of the stimulant and one or more excipients coated on an inert pellet/bead, ii) sustained release (SR) layer and iii) a delayed release (DR) layer.

In a further embodiment, the present invention provides a modified release composition comprising i) a core component comprising CNS stimulant and one or more pharmaceutically acceptable excipients or the core can consist essentially of the stimulant and one or more excipients coated on an inert pellet/bead, wherein the core is substantially free of a disintegrant or pore-forming agent; ii) sustained release (SR) layer and iii) a delayed release (DR) layer.

In a further embodiment, the sustained release component of a modified release dosage is provided as a coating over a drug containing core beads/pellets as described herein.

In yet another embodiment, the Pharmaceutical solid oral modified release composition of present invention provides a composition comprising i) a core component comprising CNS stimulant and one or more pharmaceutically acceptable excipients ii) sustained release (SR) layer coated over a drug containing core, wherein sustained release layer comprising a polymer and one or more pharmaceutical acceptable excipients and iii) a delayed release (DR) layer.

In yet another embodiment, the present invention provides a solid oral modified release composition comprising i) a core comprising CNS stimulant and one or more pharmaceutically acceptable excipients or the core can consist essentially of the stimulant and one or more excipients coated on an inert pellet/bead ii) an inner sustained release (SR) layer coated over a drug containing core, wherein sustained release layer comprising a polymer and one or more pharmaceutical acceptable excipients, and iii) an outer delayed release (DR) layer enclosing the sustained release layer; wherein the disclosed composition is prepared into a suitable dosage form with one or more pharmaceutically acceptable excipients.

In yet another embodiment, the modified release composition of present invention provides a composition comprising i) a core component comprising CNS stimulant and one or more pharmaceutically acceptable excipients ii) an inner sustained release (SR) layer coated over a drug containing core, wherein sustained release layer comprising a polymer and one or more pharmaceutical acceptable excipients, further preferable polymers include but are not limited to water insoluble and water insoluble pH dependent polymers and iii) an outer delayed release (DR) layer.

In another embodiment, the present invention provides a modified release composition comprising: i) a core comprising CNS stimulant and one or more pharmaceutically acceptable excipients; ii) an inner sustained release (SR) layer coated over the drug containing core, the sustained release layer comprising one or more polymers selected from water insoluble and water insoluble pH dependent polymers and one or more pharmaceutically acceptable excipients; and iii) an outer delayed release (DR) layer enclosing the sustained release layer.

In another embodiment, the sustained release layer includes a combination of water-insoluble polymer and water-insoluble pH dependent polymer. The rate of dissolution of the sustained release layer can be controlled by adjusting the ratio of water insoluble polymers to water-insoluble pH dependent polymer. The weight ratio of water insoluble polymer to water-insoluble pH dependent polymer can be adjusted, for example and without limitation, from 90:10 to 10:90, from 80:20 to 20:80, from 75:25 to 25:75, from 70:30 to 30:70, from 67.5:33.5 to 33.5:67.5 from 60:40 to 40:60, from 56:44 to 44:56, or to 50:50.

In yet another embodiment, the modified release composition of the present invention provides a composition comprising CNS stimulant and one or more pharmaceutically acceptable excipients, wherein CNS stimulants include but are not limited to methylphenidate or amphetamine containing active moieties, admixed with sustained release polymers, such as water-insoluble polymers, water-insoluble pH-dependent polymers to form sustained release matrix granules; further processed and enclosed by an outer delayed release (DR) layer.

In yet another embodiment, the present invention provides a modified release composition comprising i) a core comprising CNS stimulant and one or more pharmaceutically acceptable excipients ii) an inner sustained release (SR) layer coated over the drug containing core, the sustained release layer comprising one or more polymers selected from water insoluble polymers and water insoluble pH dependent polymers such as Ethyl cellulose, hydroxypropyl methylcellulose phthalate, and hydroxypropyl methylcellulose acetate succinate, a cellulose ester (such as cellulose acetate butyrate (CAB), cellulose acetate phthalate), an acrylate-based polymer or copolymer, i.e., an acrylic acid or acrylic acid ester polymer or copolymer (an “acrylate” polymer) and polyvinyl acetate-based polymers and one or more other pharmaceutical acceptable excipients and an outer delayed release layer enclosing the inner sustained release layer.

In yet another embodiment, the present invention provides a modified release composition comprising i) a core comprising CNS stimulant and one or more pharmaceutically acceptable excipients ii) an inner sustained release (SR) layer coated over a drug containing core, wherein sustained release layer comprising a polymer and one or more excipients, wherein preferable polymers include but are not limited to water insoluble polymers, water insoluble pH dependent polymers and iii) an outer delayed release (DR) layer enclosing the sustained release layer, wherein delayed release layer comprising at least one enteric polymer such as an acrylic polymer, acrylic copolymer, methacrylic polymer or methacrylic copolymer, a plasticizer, and optionally a surfactant and one or more other pharmaceutical acceptable excipients.

In yet another embodiment, the present invention provides a modified release composition comprising i) a core comprising CNS stimulant and one or more pharmaceutically acceptable excipients or the core can consist essentially of the stimulant and one or more excipients coated on an inert pellet or bead ii) an inner sustained release (SR) layer coated over a drug containing core, wherein sustained release layer comprising a polymer and one or more excipients, wherein preferable polymers include but are not limited to water insoluble polymers and water insoluble pH dependent polymers such as Ethyl cellulose, hydroxypropyl methylcellulose phthalate, and hydroxypropyl methylcellulose acetate succinate, a cellulose ester (such as cellulose acetate butyrate (CAB), cellulose acetate phthalate), an acrylate-based polymer or copolymer, i.e., an acrylic acid or acrylic acid ester polymer or copolymer (an “acrylate” polymer) and polyvinyl acetate-based polymers and iii) an outer delayed release (DR) layer enclosing the sustained release layer.

In yet another embodiment, the present invention provides a solid oral modified release composition comprising i) a core comprising CNS stimulant and one or more pharmaceutically acceptable excipients or the core can consist essentially of the stimulant and one or more excipients coated on an inert bead or pellet ii) an inner sustained release (SR) layer coated over a drug containing core, wherein sustained release layer comprising one or more polymers but are not limited to water insoluble polymers, water insoluble pH dependent polymers and a plasticizer such as glycerol, propylene glycol, low molecular weight polyethylene glycols, phthalate derivatives like dimethyl, diethyl and dibutyl phthalate, Citrate derivatives such as tributyl, triethyl, acetyltributyl citrate, at levels of from 1% to 60% of the combined weight of the polymers and one or more other pharmaceutical acceptable excipients and iii) an outer delayed release (DR) layer enclosing the sustained release layer.

In yet another embodiment, the present invention provides a modified release composition comprising i) a core comprising CNS stimulant and one or more pharmaceutically acceptable excipients or the core can consist essentially of the stimulant and one or more excipients coated on an inert pellet/bead ii) an inner sustained release (SR) layer coated over a drug containing core, wherein sustained release layer comprising combination of one or more polymers, wherein preferable combination of polymers include but are not limited to water insoluble and water insoluble pH dependent polymers, a plasticizer and one or more other additives to the coating can include but are not limited to surfactants, lubricants, anti-tacking agents etc and iii) an outer delayed release (DR) layer enclosing the sustained release layer.

In another embodiment, the present invention provides a modified release composition comprising i) a core comprising CNS stimulant and one or more pharmaceutically acceptable excipients ii) an inner sustained release (SR) layer coated over a drug containing core and iii) an outer delayed release (DR) layer enclosing the sustained release layer, wherein delayed release layer comprising one or more polymers, a plasticizer and one or more pharmaceutical acceptable excipients

In yet another embodiment, the oral modified release composition of present invention provides a composition comprising i) a core component comprising CNS stimulant and one or more pharmaceutically acceptable excipients ii) an inner sustained release (SR) layer coated over a drug containing core, wherein sustained release layer comprising one or more polymers and one or more pharmaceutical acceptable excipients and iii) an outer delayed release (DR) layer enclosing the sustained release layer, wherein delayed release layer comprising one or more enteric polymers, one or more plasticizers and one or more pharmaceutical acceptable excipients.

In yet another embodiment, the present invention provides a modified release composition comprising i) a core comprising CNS stimulant and one or more pharmaceutically acceptable excipients ii) an inner sustained release (SR) layer coated over a drug containing core, wherein sustained release layer comprising one or more polymers, optionally a plasticizers and one or more pharmaceutical acceptable excipients and iii) an outer delayed release (DR) layer enclosing the sustained release layer, further at least the delayed release layer includes one or more polymers such as an acrylic polymer, acrylic copolymer, methacrylic polymer or methacrylic copolymer, one or more plasticizers and one or more pharmaceutical acceptable excipients.

In yet another embodiment, the present invention provides a modified release composition comprising i) a core comprising CNS stimulant and one or more pharmaceutically acceptable excipients ii) an inner sustained release (SR) layer coated over a drug containing core, wherein sustained release layer comprising one or more polymers, optionally a plasticizers and one or more pharmaceutical acceptable excipients and iii) an outer delayed release (DR) layer enclosing the sustained release layer, further at least the delayed release layer includes one or more enteric polymers, a plasticizer, a surfactant and one or more pharmaceutical acceptable excipients.

In yet another embodiment, the present invention provides a modified release pharmaceutical composition comprising i) a core comprising CNS stimulant and one or more pharmaceutically acceptable excipients ii) an inner sustained release (SR) layer coated over a drug containing core, wherein sustained release layer comprising one or more polymers, optionally a plasticizers and one or more pharmaceutical acceptable excipients and iii) an outer delayed release (DR) layer enclosing the sustained release layer, further at least the delayed release layer includes one or more polymers such as an acrylic polymer, acrylic copolymer, methacrylic polymer or methacrylic copolymer, one or more plasticizers such as mono- and diglycerides, dibutyl sebacate, acetyltributylcitrate, triethyl citrate, acetyl triethyl citrate, a surfactant and one or more pharmaceutical acceptable excipients.

In yet another embodiment, the modified release pharmaceutical composition of the current invention is combined with one or more pharmaceutically acceptable excipients and prepared into a suitable dosage form.

In certain embodiment, the present invention provides a coated bead comprising: (i) a granule or Pellet comprising an active pharmaceutical ingredient comprising a central nervous system stimulant (e.g., the granule can comprise a granule substrate in admixture with the active pharmaceutical ingredient or the active pharmaceutical ingredient could be coated over the granule substrate); and one or more pharmaceutical acceptable excipients (ii) an inner sustained release layer coated over the drug containing granule or pellet, the inner layer being configured to permit substantially sustained release of the active pharmaceutical ingredient comprised therein (iii) an outer layer coated over an inner sustained release layer, the outer layer being present in an amount sufficient to substantially delay release of the active pharmaceutical ingredient until after the drug is administered 4 to 12 hours.

In another embodiment, the present invention provides a coated bead comprising: (i) a granule or Pellet comprising an active pharmaceutical ingredient comprising a central nervous system stimulant (e.g., the granule can comprise a granule substrate in admixture with the active pharmaceutical ingredient or the active pharmaceutical ingredient could be coated over the granule substrate); and one or more pharmaceutical acceptable excipients (ii) an inner sustained release layer coated over the drug containing granule or pellet, wherein sustained release layer comprising one or more polymers such as water insoluble, water insoluble pH dependent polymer, a plasticizer, and optionally a surfactant (iii) an outer layer coated over an inner sustained release layer, wherein delayed release layer comprising at least one enteric polymer such as an acrylic polymer, acrylic copolymer, methacrylic polymer or methacrylic copolymer, a plasticizer, and optionally a surfactant and one or more other pharmaceutical acceptable excipients.

In another embodiment, the present invention provides a modified release pharmaceutical composition comprising coated bead comprising: (i) a granule or Pellet comprising an active pharmaceutical ingredient comprising a central nervous system stimulant (e.g., the granule can comprise a granule substrate in admixture with the active pharmaceutical ingredient or the active pharmaceutical ingredient could be coated over the granule substrate); wherein preferable active pharmaceutical agent is Methylphenidate or Amphetamine containing active moieties and one or more pharmaceutical acceptable excipients, (ii) an inner sustained release layer coated over the drug containing granule or pellet, wherein sustained release layer comprising combination of water insoluble and water insoluble pH dependent polymer and a plasticizer and one or more pharmaceutical acceptable excipients, (iii) an outer layer enclosed an inner sustained release layer, wherein delayed release layer comprising at least one enteric polymer such as methacrylic polymer or methacrylic copolymer, a plasticizer, a surfactant and one or more other pharmaceutical acceptable excipients.

In yet another embodiment, the present invention provides a modified release pharmaceutical composition comprising coated beads or pellets, can be compressed in to suitable dosage form like tablets or minitablets.

Further, the present invention provides a modified release pharmaceutical composition comprising coated beads or pellets, are preferably formulated as a single multilayer coated bead into an oral solid pharmaceutical composition, preferably in the form of a capsule.

In another embodiment, The compositions of the present disclosure can also include one or more functional excipients such as lubricants, anti-tacking agents, anti-static agents, thermal lubricants, antioxidants, buffering agents, alkalinizing agents, binders, diluents, sweeteners, chelating agents, colorants, flavorants, surfactants, solubilizers, wetting agents, stabilizers, hydrophilic polymers, hydrophobic polymers, waxes, lipophilic materials, absorption enhancers, preservatives, absorbents, cross-linking agents, bioadhesive polymers, retardants, pore formers, and fragrance.

Examples of these pharmaceutically acceptable excipients in the component of the current invention include, but are not limited to, binders and fillers: microcrystalline cellulose, silicified microcrystalline cellulose, polyvinylpyrrolidone, hydroxypropyl cellulose, starch, pregelatinized starch, starch paste, lactose, mannitol, sorbitol, xylitol, sucrose, calcium phosphate, calcium carbonate, ethylcellulose, methylcellulose, and Acacia; lubricants (or) anti-tacking agents: talc, fumed silicon dioxide, colloidal silica, titanium dioxide, kaolin, magnesium stearate, calcium stearate, stearic acid, hydrogenated vegetable oils; surfactants: Polysorbate 80, sorbitan monooleate, polyoxymer and sodium lauryl sulfate.

### EXAMPLES

The invention is further illustrated by the following examples which are provided to be exemplary of the invention and do not limit the scope of the invention. While the present invention has been described in terms of its specific embodiments, certain modifications and equivalents will be apparent to those skilled in the art and are intended to be included within the scope of the present invention.

The active ingredient specified in the example is non-limiting and can be replaced with other active salts of CNS Stimulants and combinations thereof.

### Example 1

A batch of once daily controlled release methylphenidate composition in accordance with the present invention is prepared by methylphenidate cores containing approximately 34.8% of methylphenidate HCl.

### (a) Core

| S. | | |
|---|---|---|
| No | Drug Bead | Percentage |
| 1 | Microcrystalline cellulose (Cellets ® 500) | 34.8 |
| 2 | Methylphenidate HCl | 18.9 |
| 3 | Hydroxy Propyl Cellulose EF | 4.3 |
| 4 | Polyethylene glycol 400 | 0.3 |
| | Total | 58.3 |

The drug solution is prepared by mixing 18.9% of methylphenidate HCl, 4.3% of hydroxypropyl cellulose EF and 0.3% of PEG 400 in a container for at least 60 minutes and coating the beads by spraying the solution of methylphenidate hydrochloride to form drug beads.

### (b). Sustained Release Coating

| S. | | |
|---|---|---|
| No | SR Bead | Percentage |
| 1 | Methylphenidate beads | 58.3 |
| 2 | Cellulose Acetate Butyrate (CAB) | 5.9 |
| 3 | Hypromellose Acetate Succinate (HPMCAS) | 1.5 |
| 4 | Acetylbutyl Citrate | 1.9 |
| | Total | 67.6 |

The sustained release coating solution is prepared by combining 5.9% Cellulose Acetate Butyrate, 1.5% Hypromellose Acetate Succinate, and 1.9% Acetylbutyl Citrate in a container for at least 60 minutes and spraying the coating solution over the methylphenidate beads.

### (c). Delayed Release Coating

| S. | | |
|---|---|---|
| No | DR Coating | Percentage |
| 1 | Methacrylic Acid and Methyl Methacrylate | 27.2 |
| | Copolymer, NF (Eudragit ® S 100) | |
| 2 | Powder Silica Gel (Syloid ® 244FP) | 2.6 |
| 3 | Dibutyl Sebacate | 2.6 |
| | Total | 100 |

The delayed release coating solution is prepared by mixing 27.2% Methacrylic Acid and Methyl Methacrylate Copolymer, NF (Eudragit S 100), 2.6% Powder Silica Gel (Syloid 244FP) and 2.6% Dibutyl Sebacate in a container for at least 60 minutes, spraying the coating solution onto the methylphenidate SR beads to form delayed-release (DR) beads. The DR beads are finally compressed into tablets or filled into capsules.

### Example 2

Methylphenidate Hydrochloride's modified release (MR) composition is made up of single population beads that contain drug beads coated with sustained-release layer, followed by a delayed-release layer. The final delayed-release beads were combined with one or more additional excipients to create an appropriate dosage form; such as tablet or capsule.

| | |
|---|---|
| Ingredients | Percentage |

Drug Bead

| | |
|---|---|
| Micro crystalline cellulose (Cellets ® 500) | 31.1 |
| Methylphenidate HCl | 18.3 |
| Opadry clear | 4 |
| Total | 53.4 |

SR Bead

| | |
|---|---|
| Methylphenidate beads | 53.4 |
| Ethylcellulose EC10 | 9.4 |
| Hypromellose Acetate Succinate | 1.8 |
| Acetylbutyl Citrate | 2 |
| Total | 66.6 |

DR Coating

| | |
|---|---|
| Methacrylic Acid and Methyl Methacrylate | 27 |
| Copolymer, NF (Eudragit ® S 100) | |
| Imwitor ® (mono/di glycerides) | 2.7 |
| Dibutyl Sebacate | 2.7 |
| Polysorbate 80 | 1 |
| Total | 100 |

31.1% of microcrystalline cellulose (Cellets 500) beads were coated with the drug solution prepared by adding 18.3% of methylphenidate HCl to an aqueous binder solution, preferably a HPMC solution (Opadry Clear) in a fluid bed granulator and then dried to form drug beads. Sustained release Beads are produced by coating drug beads with the sustained-release coating solution (i.e., a mixture of water-insoluble polymers and plasticizer). The DR beads are produced by applying a layer of delayed release membrane coating (i.e., a mixture of delayed release polymer, plasticizer, and surfactant) over SR beads. The final DR beads are then filled into capsule shells at an appropriate ratio, will produce the target in vitro release profile.

### Example 3: Methylphenidate Hydrochloride's Modified Release (MR) Formulation Consists of Matrix-Type Sustained Release Formulations, Followed by a Delayed-Release Layer

| | |
|---|---|
| Ingredients | Percentage |

Intragranular

| | |
|---|---|
| Micro crystalline cellulose | 31.5 |
| Methylphenidate HCl | 18.5 |
| Hydroxypropyl methylcellulose | 4.0 |
| Total | 54 |

SR Pellet

| | |
|---|---|
| Cellulose Acetate Butyrate | 5.5 |
| Hydroxypropyl methylcellulose phthalate | 1.5 |
| Acetyl triethyl citrate | 2.0 |
| Talc | 3.0 |
| Purified water | qs |
| Total | 66 |

DR Coating

| | |
|---|---|
| Methacrylic Acid and Methyl Methacrylate | 28 |
| Copolymer, NF (Eudragit ® S 100) | |
| Imwitor ® (mono/di glycerides) | 3.0 |
| Dibutyl Sebacate | 3.0 |
| Purified water | qs |
| Total | 100.0 |

Matrix-type sustained release formulations were prepared by blending intragranular materials, spray drying the blend with a polymeric solution formed by combining the sustained-release coating polymers, surfactant and a plasticizer in water to form granules. These granules are compressed to form sustained release minitablets and further coated by a delayed release layer.

### Example 4

The coated amphetamine beads containing compositions comprising amphetamine beads, sustained release coating over amphetamine beads and delayed release coating over sustained release beads.

| | |
|---|---|
| Ingredients | Percentage |

Drug Bead

| | |
|---|---|
| Micro crystalline cellulose (Cellets ® 500) | 34.5 |
| Amphetamine | 15.6 |
| Opadry Clear | 4.5 |

SR Bead

| | |
|---|---|
| Cellulose Acetate Butyrate | 6.5 |
| Hypromellose Acetate Succinate | 1.8 |
| Triethyl citrate | 2.2 |
| Purified water | qs |

DR Coating

| | |
|---|---|
| Methacrylic Acid and Methyl Methacrylate | 28.2 |
| Copolymer, NF (Eudragit ®S 100) | |
| Imwitor ® (mono/di glycerides) | 2.8 |
| Dibutyl Sebacate | 2.8 |
| Polysorbate 80 | 1.1 |
| Purified water | qs |
| Total | 100 |

Modified Release (MR) Capsules of Amphetamine Composition Containing Coated Beads were prepared by adding 15.6% Amphetamine to an aqueous solution, preferably an HPMC solution (Opadry Clear), mixing thoroughly for at least an hour, and spraying on to microcrystalline cellulose (Cellets 500) inert beads to form Amphetamine drug beads. The sustained-release coating solution was sprayed over the amphetamine beads prepared by adding 6.5% cellulose acetate butyrate, 1.8% hypromellose acetate succinate, and 2.2% triethyl citrate in a container for at least 60 minutes to form sustained-release beads. The SR beads are coated by delayed release coating layer prepared by adding 28.2% Endragit S 100, Imwitor (mono/di glycerides), Dibutyl Sebacate, and Polysorbate 80.

We claim:

1. A modified release composition, comprising:
i) a core comprising CNS stimulant and one or more pharmaceutically acceptable excipients;
ii) an inner sustained release (SR) layer coated over the drug containing core, the sustained release layer comprising one or more polymers selected from water insoluble and water insoluble pH dependent polymers and one or more pharmaceutically acceptable excipients; and
iii) an outer delayed release (DR) layer enclosing the sustained release layer.

2. The modified release composition as claimed in claim 1, wherein the CNS stimulant selected from methylphenidate, amphetamine, or pharmaceutically acceptable salts or prodrugs thereof.

3. The modified release composition as claimed in claim 1, wherein the water insoluble polymers and water insoluble, pH dependent polymers are selected from cellulosic polymers, including ethyl cellulose, hydroxypropyl methylcellulose phthalate, and hydroxypropyl methylcellulose acetate succinate; cellulose esters; acrylate-based polymers or copolymers, including polymers or copolymers of acrylic acid or acrylic acid esters (acrylatepolymers); and polyvinyl acetate-based polymers.

4. The modified release composition as claimed in claim 3, wherein the cellulose esters are selected from cellulose acetate, cellulose acetate propionate (CAP), cellulose acetate butyrate (CAB), cellulose acetate phthalate, cellulose propionate (CP), cellulose butyrate (CB), cellulose propionate butyrate (CPB), cellulose diacetate (CDA), cellulose triacetate (CTA), cellulose acetate trimellitate (CAT).

5. The modified release composition as claimed in claim 1, wherein the sustained release layer is free of water-soluble and/or water-permeable polymers.

6. The modified release composition as claimed in claim 1, wherein the delayed release layer comprises one or more enteric polymers selected from the group consisting of acrylic polymer, acrylic copolymer, methacrylic polymer and methacrylic copolymer, or mixtures thereof.

7. The modified release pharmaceutical composition as claimed in claim 1, further comprising one or more pharmaceutically acceptable excipients, wherein the composition is formulated as capsules, tablets, beads, granules, powders, sachets, pouches, sprinkles, multi-unit particulate systems (MUPS), pellets, spheres, particles, or mini-tablets.
